(19) United States
(12) Patent Application Publication
(10) Pub. No.: US 2005/0177067 A1
(43) Pub. Date: Aug. 11, 2005

(54) System and method for urodynamic evaluation utilizing micro-electronic mechanical system

(76) Inventors: Michael R. Tracey, Branchburg, NJ (US); Anthony DiUbaldi, Jackson, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/043,830

(22) Filed: Jan. 26, 2005

Related U.S. Application Data
(60) Provisional application No. 60/543,722, filed on Feb. 11, 2004.

(51) Int. Cl. A61B 5/00

(52) U.S. Cl. 600/561; 128/903

(57) ABSTRACT

An implantable urodynamic system is provided one embodiment of which includes a power source, at least one sensor for sensing at least one physiological property, a data transmission device for transmitting data representing the at least one sensed physiological property to an exterior of the patient's bladder, and a collapsible housing containing the power source and the at least one sensor therein. The collapsible housing has a collapsed configuration sized for insertion through the patient's urethra and into the patient's bladder, and an expanded configuration sized to remain within the bladder, but be unable to pass from the bladder into the urethra.

### CROSS-REFERENCE TO RELATED APPLICATION

This application claims the benefit of U.S. Provisional Application Ser. No. 60/543,722 filed on Feb. 11, 2004.

### BACKGROUND OF THE INVENTION

1. Field of the Invention

The present invention relates generally to devices and methods for urodynamic evaluation, and more particularly, to such a system and method that utilizes micro-electronic mechanical system (MEMS) technology.

2. Background Discussion

Women account for more than 11 million incontinence cases. One type of incontinence is stress urinary incontinence (SUI), where women experience involuntary loss of urine during normal daily activities and movements, such as laughing, coughing, sneezing and regular exercise. SUI may be caused by a functional defect of the tissue or ligaments connecting the vaginal wall with the pelvic muscles and pubic bone. Common causes include repetitive straining of the pelvic muscles, childbirth, loss of pelvic muscle tone, and estrogen loss. Such a defect results in an improperly functioning urethra. Unlike other types of incontinence, SUI is not a problem of the bladder.

Another form of incontinence is urge incontinence, which is caused by overactive bladder muscles. One example is detrusor instability, which involves spontaneous and unprovoked involuntary contractions of the detrusor muscle (the muscles that make up the bladder wall) that cannot be suppressed during filling of the bladder.

Incontinence in general, be it SUI or urge incontinence, is both embarrassing and unpredictable, and many women with SUI avoid an active lifestyle and shy away from social situations.

In order to treat urinary incontinence, it must first be understood which type of incontinence the patient is suffering from, and the physical causes for the incontinence. Only then can the proper treatment be prescribed. Many types of urodynamic systems and tests are currently available to try to assess the type and causes of incontinence. These systems can be broadly categorized in two ways: office based systems and ambulatory systems. Office based systems are designed for use in a doctor's or clinician's office. Many of these systems involve invasive testing using catheters and the like. Ambulatory systems are designed to capture data outside the office over a longer period of time such as 1-2 days. Known ambulatory systems for urodynamic measurements are also invasive in that they use catheters to capture pressure data within the urethral tract or in the bladder. It is readily apparent that such known ambulatory systems are uncomfortable and invasive for the patient. Further, because the catheters are inter-dwelling, they are prone to movement or migration over time as the patient moves around. In addition, they may not accurately capture typical daily occurrences, as the patient is, due to the discomfort, prone to move less and engage in less activities than normal while undergoing the assessment. Finally, the invasive catheters may also interfere with true physiological responses, as they can irritate the internal tissues/organs through which they are inserted. Thus, migration of the pressure sensors and their invasive nature limits the reliability and usefulness of the data.

There has been interest generated around developing implantable microdevices for use in medical applications. Some of this attention has focused on Micro Electro Mechanical Systems (MEMS), which is a class of small devices that integrates tiny mechanical and electrical components on a silicon chip. One example of the application of microdevices in the medical field is an implantable device that enables real-time monitoring of blood glucose by an implantable sensor, and in response allows automated insulin delivery (see e.g. European Patent No. 1048264). Microdevices that automatically deliver dosages of other chemicals or pharmaceuticals have also been contemplated (see e.g., U.S. Pat. Nos. 5,558,640, 6,438,407 and 6,183,461), as have microdevices for use in ambulatory urodynamics. See Siwapornsathain, E., Lal, A., Binard, J., “Telemetry and Sensor Platform for Ambulatory Urodynamics,” Proceedings of the 2^{nd} Annual International IEEE-EMBS Special Topic Conference on Microtechnologies in Medicine & Biology, Madison, Wis., May, 2002. Although the concept of implantable devices for ambulatory urodynamics is revealed in the previously cited article, the device described therein has little if any practical value. The described device is too large for suitable use, and does not capture sufficient data to assess incontinence or its cause(s). For example, the device contemplates capturing only bladder pressure, but only provides a device that captures a range of pressures and at a resolution such that they have no clinical value.

The present application describes an improved and robust implantable device and system that effectively captures ambulatory urodynamic data for assessment of urinary incontinence.

### SUMMARY OF THE INVENTION

The present invention provides an implantable urodynamic system for implanting within a patient's body including a power source, at least one sensor for sensing at least one physiological property, a data transmission device for transmitting data representing the at least one sensed physiological property to an exterior of the patient's bladder, and a collapsible housing containing the power source and the at least one sensor therein. The collapsible housing has a collapsed configuration sized for insertion through the patient's urethra and into the patient's bladder, and an expanded configuration sized to remain within the bladder, but be unable to pass from the bladder into the urethra.

The at least one sensor may be a pressure sensor for sensing pressure within the bladder, and the power source and at least one sensor may further be encapsulated within a sealed protective cover, which itself may be made of silicone.

In one embodiment, the sealed system has a length less than about 20 mm and a height less than about 12 mm in the collapsed state, and according to another embodiment, the collapsible housing is comprised of nitinol.

In yet another embodiment, the data transmission device further includes a data capture element for capturing data representing the at least one sensed physiological property from the at least one sensing element, and a data transmission element for transmitting said captured data. The collapsible housing may be made of a metal wherein the data transmission element forms part of the collapsible housing. In an alternate embodiment, the data tranmission element is an antennae extending outwardly from the collapsible housing.

A further embodiment includes at least two pressure sensing elements and a tail element extending outwardly from the collapsible housing. A first of the sensing elements is positioned within the collapsible housing, and a second of the sensing elements is positioned on the tail element.

In yet another embodiment, when the collapsible housing is positioned within the bladder in the expanded configuration, the tail element extends from the bladder into the urethra. In such an embodiment, the first of the sensing elements may sense bladder pressure, and the second of the sensing element may sense urethral pressure. In an alternative embodiment, the first of the sensing elements may sense bladder pressure, and the second of the sensing elements may sense the presence of fluid. In yet another alternative embodiment, the first of the sensing elements may sense bladder pressure, and the second of the sensing elements may sense fluid velocity.

Also provided is a urodynamic system including a first implantable device sized for implantation within a patient's bladder. The first device includes a power source, at least one sensor for sensing a physiological property within the bladder, and a data storage element for storing data representing the physiological property sensed by the sensor. The system further includes a second implantable device sized for implantation within the patient's vagina, and including a power source, at least one pressure sensor for sensing pressure within the vaginal canal, and a data storage element; and a data retrieval device for, following removal of the first and second implantable devices from the patient's body, retrieving and manipulating data from the first and second data storage elements. In one embodiment, the second implantable device is encapsulated within a pliable casing dimensioned to securely but removably engage the vaginal walls. The pliable casing may be made of cotton. According to one embodiment, the at least one sensor of the first implantable device senses bladder pressure.

In another embodiment, the system further includes a collapsible housing containing the first implantable device. The collapsible housing has a collapsed configuration sized for insertion through the patient's urethra and into the patient's bladder, and an expanded configuration sized for insertion within the bladder, but to prevent it's passage from the bladder into the urethra.

The present invention also provides a urodynamic system including a first implantable device sized for implantation within a patient's bladder and a second implantable device sized for implantation within a patient's bladder. The first device includes a power source, at least one sensor for sensing a physiological property within the bladder, and a data transmission device for transmitting data representing the sensed physiological property to a point external of the patient's bladder. The second device includes a power source, at least on sensor for sensing a pressure within the patient's vaginal canal, and a data transmission device for transmitting data external of the patient's vaginal canal. The system may further include a data processing device for receiving and processing transmitted data received from the first and second implantable devices.

These and other features and advantages of the present invention will become apparent from the following more detailed description, when taken in conjunction with the accompanying drawings which illustrate, by way of example, the principles of the invention.

### BRIEF DESCRIPTION OF THE DRAWINGS

FIG. 1 illustrates electronic components, including an internal data storage device, of an implantable device according to one embodiment of the present invention;

FIG. 1a illustrates electronic components, including an external data storage device, of an implantable device according to an alternate embodiment of the present invention;

FIG. 2a illustrates an implantable device according to one embodiment of the present invention including an expandable cage in its non-expanded state;

FIG. 2b illustrates the device of FIG. 2a with the expandable cage in the expanded state;

FIG. 3 illustrates an implantable device according to yet another embodiment of the present invention without an expandable cage;

FIGS. 4a-4c illustrate various steps of deployment of an implantable device according to one embodiment of the present invention;

FIGS. 5a and 5b are schematic diagrams illustrating flow of data in alternate embodiments of the present invention;

FIG. 6 illustrates one embodiment of an implantable device deployed within the bladder and having a tail extending into the urethra;

FIG. 7 is a schematic diagram illustrating an external data storage element receiving input data from an implantable device and from an input device;

FIG. 8 illustrates an implantable system according to the present invention including first and second implantable devices;

FIG. 9 illustrates an implantable device that incorporates a sensor on a tail element; and

FIG. 9a illustrates an implantable device that incorporates multiple sensors on multiple tail elements.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. For example, although the present invention is described in detail in relation to the female urinary system, it is to be understood that it can be readily adapted for use in the male urinary system. Further, the inventive principles, apparatus and methods disclosed herein may also have application to assessing functionality in other areas, such as coronary or pulmonary functionality.

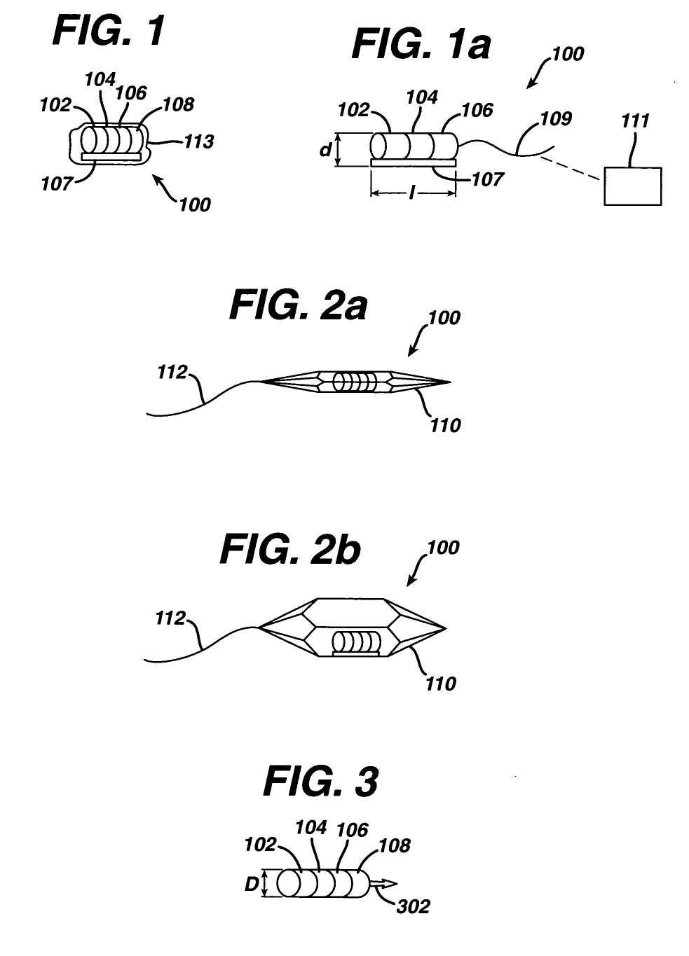

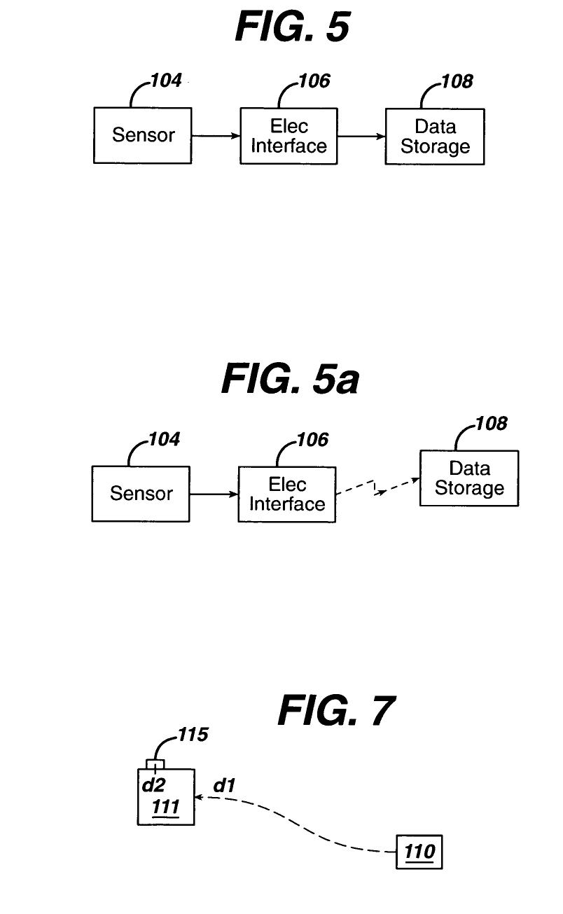

Various embodiments and/or elements of an implantable urodynamic system 100 according to the present invention is shown schematically in FIGS. 1, 1a, 2a and 2b, and will be described in conjunction with intended implantation into a patient's bladder. The system includes multiple electronic components including a power source 102, one or more sensor components 104, and an electronic interface 106, each of which are electrically coupled to one another and mechanically mounted on a printed circuit board 107 in a manner well known in the art. The one or more sensor components 104 sense predetermined physiological properties within the body, and transmit signals or data representing such properties to the electrical interface 106. In one embodiment, the system further includes a data storage element 108 for storing data correlating to the data representing the physiological properties. In an alternate embodiment shown in FIG. 1 a, rather than a data storage element, the system further includes a transmitter 109 for transmitting data external of the patient's body, which is subsequently captured and stored on an external data storage device 111. FIGS. 5 and 5a demonstrate schematically the flow of data in the embodiments of FIGS. 1 and 1a respectively, with solid lines indicating transmission via hard wiring and dotted lines indicating wireless transmission. As shown in both FIGS. 2a and 2b, in one embodiment the components described above are surrounded by housing 110 or cage, which in the illustrated embodiment is a collapsible cage that will be described in more detail below.

Preferably, the system (exclusive of the housing) has an overall size of about 0.65-10 mm in diameter d, and about 0.65-10 mm in length l. In a preferred embodiment, the sensor component is a micro-miniature piezo-resistive pressure transducer for measuring pressure within a patient's bladder. A suitable transducer is an MPX series pressure sensor from Motorola of Schaumburg, Ill. Other suitable components may include the MSP430F149 microcontroller from Texas Instruments, Inc. of Dallas, Tex. that can be used to acquire, filter and store data from the pressure sensor, and power source such as any suitable biocompatible lithium battery. Although particular suitable electronic components have been named above, many others also exist and could be incorporated into the present invention. As indicated, the electronic components are preferably mounted on printed circuit board. Subsequently, the components and circuit board can be covered or encapsulated in silicone or other suitable covering 113 (as shown only in FIG. 1) to protect them from the environment, such as the fluid environment in the bladder

Referring now again to the housing 110 as illustrated in greater detail in FIGS. 2a and 2b, in a preferred embodiment the housing is a collapsible cage made of a suitable metal such as Nitonol, stainless steel, or a titanium alloy, or a suitable biocompatible polymer such as polypropylene or polyethylene terapthalate. The collapsible cage is advantageous in that it can exist in a collapsed state shown in FIG. 2a that is sufficiently small to allow insertion through the patient's urethra. Once inserted into the bladder as will be described further below, however, the cage can assume the expanded state shown in FIG. 2b, which has a size sufficiently large so that it cannot pass back into the urethra, and thus will remain in the bladder until physical removal is desired. In the illustrated embodiment, the housing or cage is preferably made of Nitinol and returns to its expanded state (FIG. 2b) when not compressed by an external force. The electrical components and printed circuit board can be mechanically affixed to the cage in any suitable manner, such as by using a biocompatible adhesive. The housing may further include a tail element 112 extending outwardly therefrom. This tail element 112 may operate as the transmitter for the device as an alternate to the transmitter configuration shown in FIG. 1a. As will be further described below, this tail element 112 may also incorporate additional sensor elements if desired.

In another embodiment, the expandable cage may be made of an absorbable material such as Ethisorb® (an absorbable synthetic composite made from polyglactin and polydioxanon) from Ethicon, Inc. of Somerville, N.J., or a combination of absorbable and non-absorbable materials. The absorbable material would preferably dissolve after a predetermined period of time, such as at least 2-3 days, so that the implantable device could be expelled from the body in a non-invasive manner after sufficient data has been gathered.

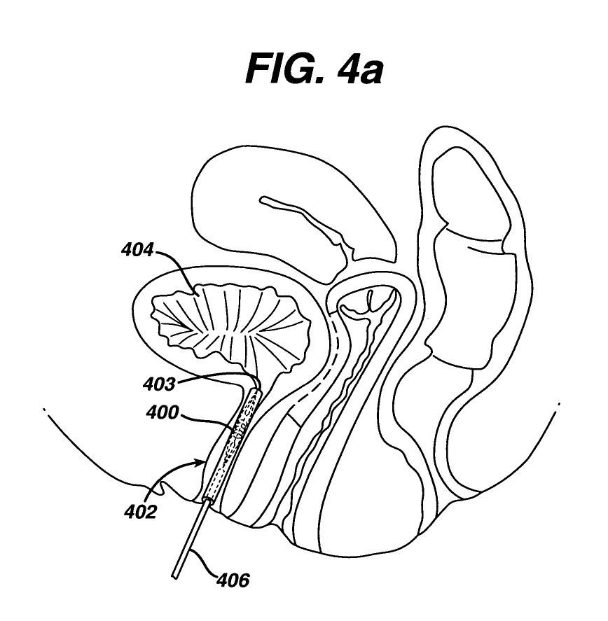

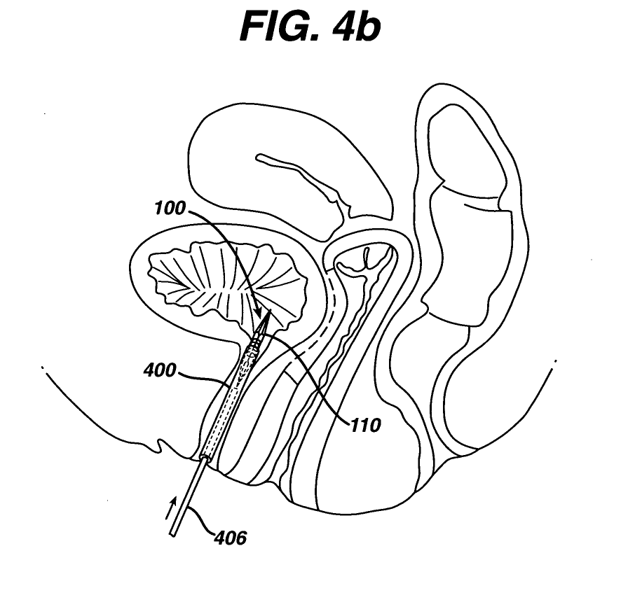

As an alternative to the collapsible cage described above, the housing could have a stable structure rather than a collapsible structure that itself has an outer diameter D that is smaller than the diameter of the urethra to allow insertion therethrough into the bladder (see FIG. 3). The housing may further have one or more projections 302, such as screw threads, barbs or the like, extending outwardly therefrom that can be attached to the sidewall of the bladder by being pushed or driven therein. In yet other alternate embodiments, the implantable device could be sutured to the bladder wall, or adhered thereto using a suitable biocompatible adhesive.

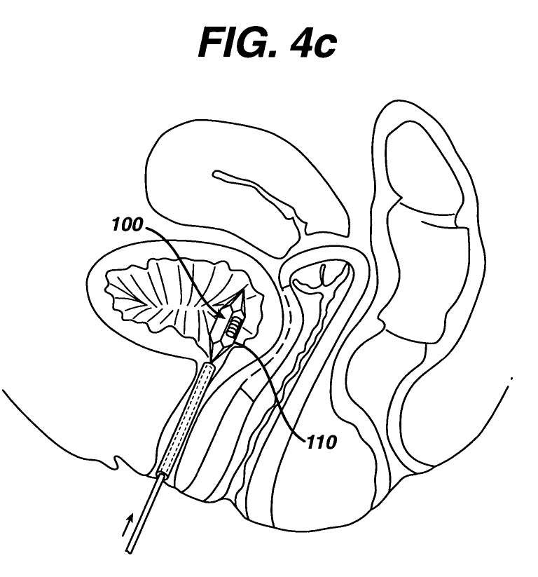

Use of the above-described device will now be described in detail. The system 100 with the housing in the compressed state is loaded into a single or multi-lumen catheter 400 as shown in FIG. 4a, which inserted through the urethra 402 until the tip or distal end 403 is positioned within the bladder 404. The catheter may be any catheter suitable for intra-urethral applications, such as a Foley catheter. Fluroroscopy, ultrasound or other similar technology known to those skilled in the art may be used to aid in delivery and placement of the implantable system within the bladder. If a multi-lumen catheter is used, other lumens may be used to fill or drain the bladder, deliver drugs, provide an access for visualization, or monitor pressure while placing the implantable system. An expulsion element 406, such as a push rod or the like is inserted into the primary lumen behind the implantable system 100, and once the distal end of the catheter is properly positioned within the bladder, the expulsion element is moved toward the distal end of the catheter in the direction of the arrow as shown in FIGS. 4b and 4c to thereby expel the implantable system 100 from the distal end of the catheter and into the bladder. As the implantable system exits the catheter, the collapsible cage 110 is no longer being held in its collapsed state, and proceeds to expand to its fully expanded state. Although use of a catheter is described, other suitable implantation methods may also be used, such as placement via the working channel in a cystoscope or similar surgical tool, or placement via laparoscopic or open surgical methods. Once deployed within the bladder, the expandable cage is dimensioned to prevent the device from being lodged in the bladder neck or otherwise passing into the urethra, but further allows urine to freely flow through it. FIG. 6 illustrates the implantable device 100 fully deployed within the bladder 404.

As mentioned above, alternate embodiments that do not employ expandable cages may also be suitable, such as that shown in FIG. 3. The method of implantation of such devices would be similar to that described above, with the expulsion element within the catheter being used to drive the projecting element 302 into the wall of the bladder to thereby anchor the device to the bladder.

The device can remain within the bladder for at least as long as is necessary to obtain the desired data. For example, the device could remain within the bladder for 1-2 days, with bladder pressure measurements being taken every ½ second. The type and frequency of bladder pressure changes can be subsequently analyzed to provide feedback to assess urinary function. For example, vesicle pressure measured over time can reveal voiding times and frequency, can provide an indication of an overactive bladder, or of bladder overfilling. In one embodiment, the sensor element(s) are designed to operate in an extended sleep mode, “waking up” at fixed intervals of time to measure pressure or the like. Once sufficient data has been gathered, the device can subsequently be removed from the bladder by inserting a catheter into the bladder to retrieve the implantable device, or using the operating channel of a cystoscope or other suitable instrument to retrieve the device. The catheter or cystoscope would be inserted into the bladder, and the device grasped and pulled back into the catheter or cystoscope channel and subsequently removed from the body.

Following data acquisition and storage, the data must then be retrieved to allow for its analysis and manipulation, preferably by uploading the data to a PC based software application. Data from the data storage element of the implantable device of FIG. 1, can be uploaded to a PC by any suitable manner, such as wirelessly, for example, via an infrared data acquisition unit such as ENDEC HSDL-7001 and an IrDA transceiver HSDL-3202 interfaced to the microprocessor, via radiofrequency acquisition, or via a hard wire connection such as through an RS232 interface. The pressure data is then formatted and displayed on the PC as pressure versus time, or in any other suitable manner.

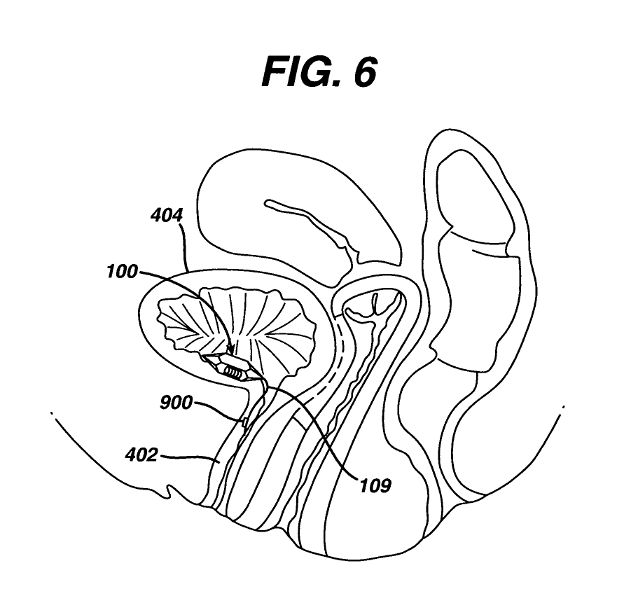

As indicated above, in the embodiment of FIG. 1a, the data from the sensor element may be transmitted external to the patient's body to an external storage element or receiver 111, such as by using well known radio frequency transmission techniques via a transmitter or antennae 109. The antennae may be any suitable conductive material, but preferably would be comprised of nitonol and integrated into the nitonol cage described above. The receiver may be a small device that would be carried by the patient and similar in size to a personal communication device. The receiver may additionally have the ability to receive other forms of input data. For example, as shown in FIG. 7, the receiver 111 may receive input data d1 from the implantable device via radiofrequency as described above, and also receive input data d2 from the patient that corresponds to external events that impact bladder pressure, such as coughing or sneezing. This second input data d2 may be input via a digital button 115 on the receiver or other input pendant, or via a digital voice recorder or the like.

An implantable device for ambulatory urodynamics has been described in its most simplest form above. The present invention, however, contemplates various other modifications and configurations. For example, the sensor components may be designed to measure any number of parameters, such as pressure, chemical composition of body fluids/tissues, temperature, electrical impedance, or fluid velocity or acceleration. Multiple different sensors measuring multiple different parameters may also be employed, with data potentially being transferred therebetween by wireless transmission or otherwise. In this manner, pH measurements and/or temperature measurements can be taken, impedance measurements can be taken for measuring flow rate for urinary leak detection, and fluid acceleration can be measured to determine the positioning of the patient (i.e., horizontal (lying down) or vertical (standing). Miniature cameras employing Complimentary Metal Oxide Semi-Conductor (CMOS) technology may also be used as a sensor element.

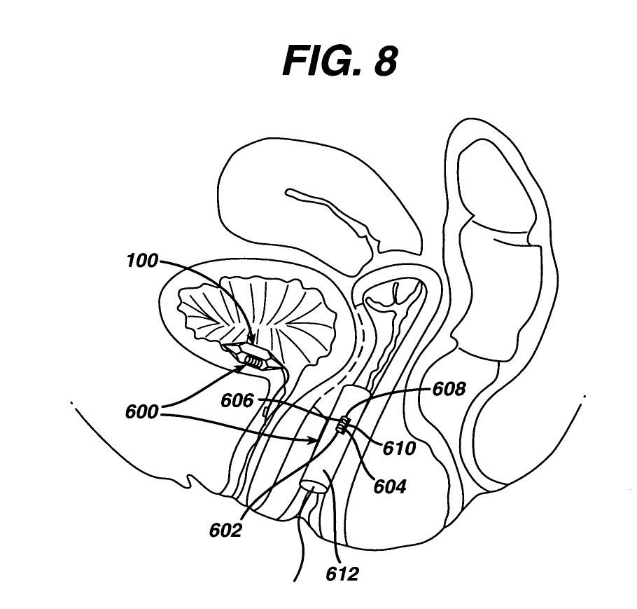

In one particularly useful embodiment shown in FIG. 8, the implantable system 600 further includes a second implantable device 602 that includes a second power source 602, a second sensor element(s) 604, a second electrical interface 606, and a second data storage element 608 (alternatively an external storage element as described above), which are similarly integrated on a printed circuit board 610. As described above with the first implantable device, the second device is preferably encapsulated in silicone or the like. The second implantable device, however, is designed for insertion into the vaginal canal of a patient, and thus is preferably encapsulated in a “tampon-like” device or casing as shown. This casing 612 is preferably simply rolled up or bound cotton, similar to a tampon. In an alternate embodiment, only one of the two implantable devices includes a data storage element, or transmits data to an external data storage element, and the other would simply wirelessly transmit its obtained pressure data to the other one. The sensor element is preferably a pressure sensor for sensing abdominal pressure from within the vagina. With the second implantable device sensing abdominal pressure, and the first implantable device sensing bladder pressure, the detrusor pressure (pressure of the muscle lining of the wall of the bladder tissue) can be determined by subtracting the bladder pressure from the abdominal pressure. Rises in detrusor pressure will occur if the patient strains, coughs, sneezes, laughs, etc., and detection of these pressures are clinically significant in the diagnosis of various bladder and lower urinary tract disease states. For example, the frequency of detrusor pressure increases provides meaningful data for assessing urge incontinence.

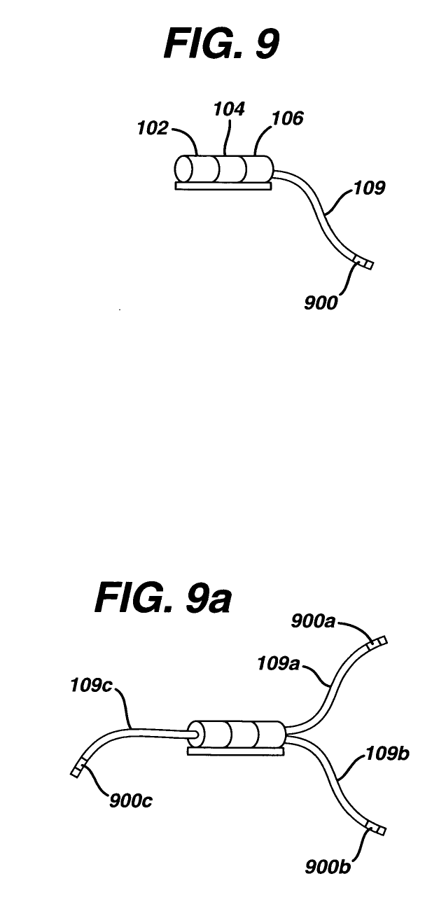

In yet another embodiment, the first implantable device that is implanted within the bladder further includes one or more additional sensors 900 that are incorporated into one or more tail elements, as shown in FIGS. 9 and 9a. In one particular implementation, the sensor(s) are leak detection sensors incorporated into a tail that is designed to extend from the device within the bladder, through the sphincter and into the urethral canal 402 as shown in FIG. 6. This sensor(s) detect the presence of fluid, and thus will detect leakage of urine such as occurs in a stress incontinent patient, while at the same time the pressure sensor within the bladder measures bladder pressure. Thus, stress incontinence episodes can be recorded by correlating time at which a rise in bladder pressure occurs concurrently with detection of fluid leakage through the urethra.

Further, multiple tail elements 109a, 109b, 109c may incorporate multiple sensor elements 900a, 900b, 900c as shown in FIG. 9a to record the pressure at different points in the bladder, and thus provide more accurate readings.

It will be apparent from the foregoing that, while particular forms of the invention have been illustrated and described, various modifications can be made without departing from the spirit and scope of the invention. Accordingly, it is not intended that the invention be limited, except as by the appended claims.

1. An implantable urodynamic system for implanting within a patient's body comprising:
a power source;
at least one sensor for sensing at least one physiological property;
a data transmission device for transmitting data representing the at least one sensed physiological property to an exterior of the patient's bladder; and
a collapsible housing containing the power source and the at least one sensor therein, the collapsible housing having a collapsed configuration sized for insertion through the patient's urethra and into the patient's bladder, and an expanded configuration sized to remain within the bladder, but be unable to pass from the bladder into the urethra.

2. The device according to claim 1, wherein the at least one sensor is a pressure sensor for sensing pressure within the bladder.

3. The device according to claim 1, wherein the power source and at least one sensor are encapsulated within a sealed protective cover.

4. The device according to claim 3, wherein the sealed protective cover is comprised of silicone.

5. The device according to claim 3, wherein the sealed system in the collapsed state has a length less than about 20 mm and a height less than about 12 mm.

6. The device according to claim 1, wherein the collapsible housing is comprised of nitinol.

7. The device according to claim 1, wherein the data transmission device further comprises a data capture element for capturing data representing the at least one sensed physiological property from the at least one sensing element, and a data transmission element for transmitting said captured data.

8. The device according to claim 6, wherein the collapsible housing is comprised of a metal, and the data transmission element forms part of the collapsible housing.

9. The device according to claim 6, wherein the data tranmission element is an antennae extending outwardly from the collapsible housing.

10. The device according to claim 1, comprising at least two pressure sensing elements and further comprising a tail element extending outwardly from the collapsible housing, wherein a first of said sensing elements is positioned within said collapsible housing, and a second of said sensing elements is positioned on said tail element.

11. The device according to claim 10, wherein when the collapsible housing is positioned within the bladder in the expanded configuration, the tail element extends from the bladder into the urethra.

12. The device according to claim 11, wherein the first of said sensing elements senses bladder pressure, and the second of said sensing element senses urethral pressure.

13. The device according to claim 11, wherein the first of said sensing element senses bladder pressure, and the second of said sensing element senses the presence of fluid.

14. The device according to claim 11, wherein the first of said sensing element senses bladder pressure, and the second of said sensing elements senses fluid velocity.

15. An urodynamic system comprising:
a first implantable device sized for implantation within a patient's bladder, the first device including a power source, at least one sensor for sensing a physiological property within the bladder, and a data storage element for storing data representing the physiological property sensed by said sensor;
a second implantable device sized for implantation within the patient's vagina, the second device including a power source, at least one pressure sensor for sensing pressure within the vaginal canal, and a data storage element;
a data retrieval device for, following removal of the first and second implantable devices from the patient's body, retrieving and manipulating data from said first and second data storage elements;

16. The system according to claim 15, wherein the second implantable device is encapsulated within a pliable casing dimensioned to securely but removably engage the vaginal walls.

17. The system according to claim 16, wherein the pliable casing is comprised of cotton.

18. The system according to claim 15, wherein the at least one sensor of the first implantable device senses bladder pressure.

19. The system according to claim 15, further comprising a collapsible housing containing the first implantable device, the collapsible housing having a collapsed configuration sized for insertion through the patient's urethra and into the patient's bladder, and an expanded configuration sized for insertion within the bladder, but to prevent it's passage from the bladder into the urethra.

20. A urodynamic system comprising:
a first implantable device sized for implantation within a patient's bladder, the first device including a power source, at least one sensor for sensing a physiological property within the bladder, and a data transmission device for transmitting data representing the sensed physiological property to a point external of the patient's bladder;
a second implantable device sized for implantation within a patient's bladder, the second device including a power source, at least on sensor for sensing a pressure within the patient's vaginal canal, and a data transmission device for transmitting data external of the patient's vaginal canal;

21. The system according to claim 20, further comprising a data processing device for receiving and processing transmitted data received from the first and second implantable devices.
